(19) United States
(12) Patent Application Publication
(10) Pub. No.: US 2022/0370821 A1
(43) Pub. Date: Nov. 24, 2022

(54) PUNCTURE DEVICE AND PHOTOTHERAPY METHOD

(71) Applicant: OLYMPUS CORPORATION, Tokyo (JP)

(72) Inventor: Tomohiko MAMIYA, Tokyo (JP)

(73) Assignee: OLYMPUS CORPORATION, Tokyo (JP)

(21) Appl. No.: 17/874,426

(22) Filed: Jul. 27, 2022

Related U.S. Application Data
(63) Continuation of application No. PCT/JP2020/005946, filed on Feb. 17, 2020.

(51) Int. Cl. A61N 5/06; A61B 8/12; A61B 8/00

(52) U.S. Cl. CPC A61N 5/0603; A61B 8/12; A61B 8/445; A61N 2005/0612; A61N 2005/063

(57) ABSTRACT

Provided is a puncture device includes: a needle tube; and a tube that is accommodated within the needle tube, is capable of accommodating an optical fiber, and is composed of a cylindrical optically-transparent material. The needle tube includes a tip member fixed to a distal end of the tube and having a blade surface at a tip thereof, and a base member that is disposed at a position where the base member covers a proximal end of the tube relative to the tip member and that is movable along a longitudinal axis of the needle tube. The base member advances to cause a distal end of the base member to abut on a proximal end of the tip member. The base member retracts to cause the distal end of the base member to move away from the proximal end of the tip member in a direction of the longitudinal axis.

### CROSS-REFERENCE TO RELATED APPLICATIONS

This is a continuation of International Application PCT/JP2020/005946, with an international filing date of Feb. 17, 2020, which is hereby incorporated by reference herein in its entirety.

### TECHNICAL FIELD

The present invention relates to puncture devices and phototherapy methods.

### BACKGROUND ART

Known technology involves injecting a test subject with a drug that specifically accumulates in cancer cells and kills the cancer cells by reacting to near-infrared light, and subsequently irradiating the cancer cells with near-infrared light by using an optical fiber inserted into the body of the test subject (e.g., see Patent Literature 1). If there are cancer cells in, for example, the pancreas located deep inside the body, an endoscope is inserted deep into the body to, for example, the stomach or the duodenum, and a needle assembly is inserted into the body via a channel in the endoscope. Then, in a state where the needle assembly has punctured the cancer cells in, for example, the pancreas, the optical fiber inserted in a needle tube of the needle assembly is made to protrude from the needle tip, and the cancer cells are irradiated with light via the optical fiber.

### CITATION LIST

### Patent Literature

### {PTL 1}

Japanese Translation of PCT International Application, Publication No. 2014-523907

### SUMMARY OF INVENTION

A first aspect of the present invention is directed to a puncture device comprising: a metallic needle tube having a longitudinal axis; and a tube that is accommodated within the needle tube, the tube being capable of accommodating an optical fiber along the longitudinal axis, the tube being composed of a cylindrical optically-transparent material. The needle tube comprises a needle tip member and a needle base member. The needle tip member is fixed to a distal end of the tube and has a blade surface at a tip of the needle tip member. The needle base member is disposed at a position where the needle base member covers a proximal end of the tube relative to the needle tip member and is movable along the longitudinal axis. The needle base member is advanced to cause a distal end of the needle base member to abut on a proximal end of the needle tip member, and the needle base member is retracted to cause the distal end of the needle base member to move away from the proximal end of the needle tip member in a direction of the longitudinal axis.

A second aspect of the present invention is directed to a puncture device comprising: a metallic needle tube having a blade surface at a distal end of the needle tube; and an optical fiber accommodated within the needle tube, the optical fiber extending along a longitudinal axis of the needle tube. The distal end of the needle tube is provided with a side hole with an opening oriented in a direction intersecting the longitudinal axis and that exposes a light radiation range in which light is emitted from the optical fiber. A distal end of the light radiation range in a direction of the longitudinal axis is positioned toward the distal end of the needle tube relative to a distal end of the side hole in the direction of the longitudinal axis, and a proximal end of the light radiation range in the direction of the longitudinal axis is positioned toward a proximal end of the needle tube relative to a proximal end of the side hole in the direction of the longitudinal axis.

A third aspect of the present invention is directed to a phototherapy method comprising: inserting an ultrasonic endoscope into an alimentary canal; rendering an irradiation site within a body by using the ultrasonic endoscope inserted in the alimentary canal; causing a needle tube accommodating a tube composed of an optically transparent material to protrude from a distal end of the ultrasonic endoscope inserted in the alimentary canal; causing the protruding needle tube to puncture a vicinity of the irradiation site; exposing a portion of the tube from the needle tube in a state where the needle tube has punctured the vicinity of the irradiation site; and transmitting light emitted from an optical fiber through the tube exposed from the needle tube to irradiate the irradiation site with the light.

### BRIEF DESCRIPTION OF DRAWINGS

FIG. 1 illustrates a state where a needle tube of a puncture device according to an embodiment of the present invention has punctured an alimentary canal.

FIG. 2 is a vertical sectional view of the puncture device in FIG. 1.

FIG. 3 is a perspective view of the puncture device in FIG. 1.

FIG. 4 is a vertical sectional view illustrating a state where a needle base member of the needle tube in FIG. 2 is in abutment with a needle tip member.

FIG. 5 is a vertical sectional view illustrating a state where the needle base member of the needle tube in FIG. 2 is positioned away from the needle tip member.

FIG. 6 is a vertical sectional view of an optical fiber accommodated within a tube in FIG. 2.

FIG. 7 is a perspective view of the puncture device in FIG. 1, as viewed from a different angle.

FIG. 8 is a plan view of a slider body and a pipe sliding section and illustrates a state where a lock button has been moved to an unlocking position.

FIG. 9 is a plan view of the slider body and the pipe sliding section and illustrates a state where the lock button has been moved to a locking position.

FIG. 10 is a plan view of the pipe sliding section in FIG. 9, as viewed in a direction parallel to a central axis.

FIG. 11 is a flowchart illustrating a phototherapy method using the puncture device in FIG. 1.

FIG. 12 illustrates a state where cancer cells are being irradiated with light by using the puncture device in FIG. 1.

FIG. 13 is a vertical sectional view of the needle tube and illustrates a tube of a puncture device according to a modification of the embodiment of the present invention.

FIG. 14 is a vertical sectional view of the needle tube and illustrates another tube of a puncture device according to a modification of the embodiment of the present invention.

FIG. 15 is a vertical sectional view illustrating a state where a stylet is fitted in the tube of a puncture device according to a modification of the embodiment of the present invention.

FIG. 16 is a vertical sectional view illustrating a state where the distal end of the needle base member of a puncture device according to a modification of the embodiment of the present invention covers the proximal end of the needle tip member while being in abutment therewith.

FIG. 17 is a vertical sectional view illustrating a state where the needle base member in FIG. 16 is positioned away from the needle tip member.

FIG. 18 is a vertical sectional view illustrating another example where the distal end of the needle base member of a puncture device according to a modification of the embodiment of the present invention covers the proximal end of the needle tip member while being in abutment therewith.

FIG. 19 is a perspective view of a puncture device according to a modification of the embodiment of the present invention.

FIG. 20 is a vertical sectional view of the optical fiber accommodated within the tube of a puncture device according to a modification of the embodiment of the present invention.

FIG. 21 is a vertical sectional view of another optical fiber accommodated within the tube of a puncture device according to a modification of the embodiment of the present invention.

FIG. 22 is a vertical sectional view of the needle tube and the optical fiber and illustrates an example of the relationship between the distance of a range in which the tube is exposed and the length of a light radiation section of the optical fiber in a configuration where the needle tube is separable.

FIG. 23 is a vertical sectional view of the needle tube and the optical fiber and illustrates an example of the relationship between the distance of the range in which the tube is exposed and the length of the light radiation section of the optical fiber in a configuration where the needle tube has a side hole.

FIG. 24 illustrates a state where the cancer cells are being irradiated with light by using a puncture device according to a modification of the embodiment of the present invention.

### DESCRIPTION OF EMBODIMENTS

A puncture device and a phototherapy method according to an embodiment of the present invention will be described below with reference to the drawings.

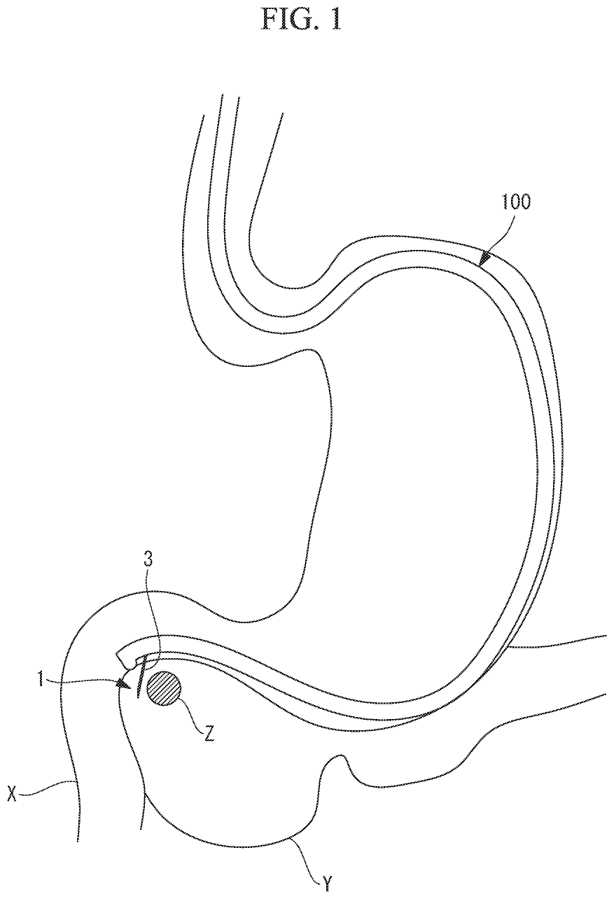

As shown in FIG. 1, a puncture device 1 according to this embodiment is to be inserted into the body (i.e., a living body) of a patient (i.e., a test subject) via a channel (not shown) provided in an ultrasonic endoscope 100. In FIG. 1, reference sign X denotes an alimentary canal, such as the stomach or the duodenum, reference sign Y denotes the pancreas, and reference sign Z denotes cancer cells (i.e., an irradiation site).

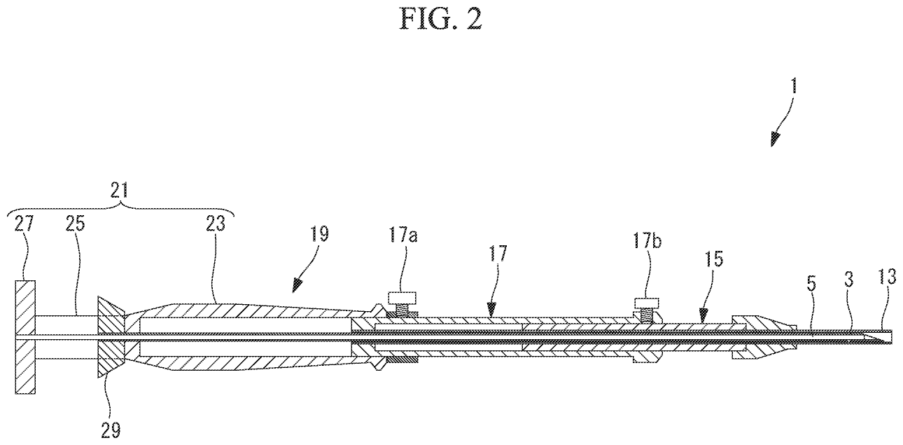

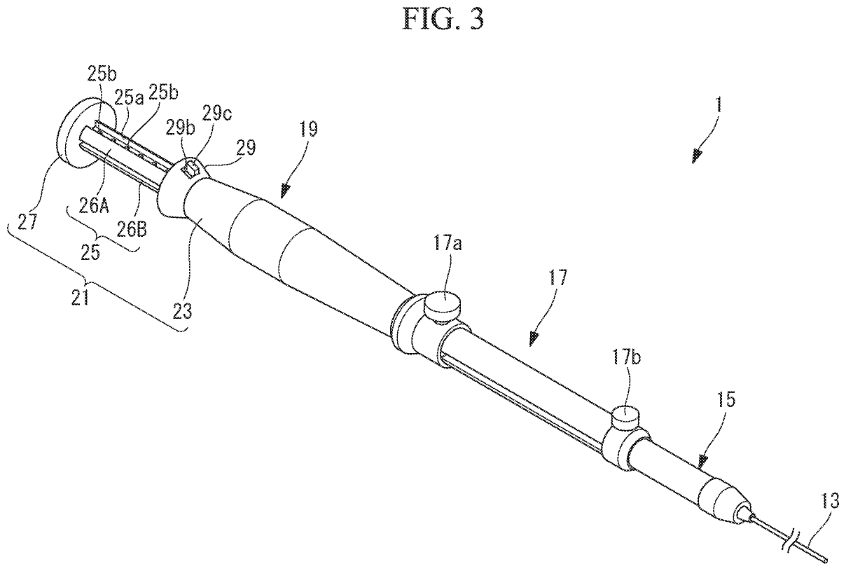

As shown in FIG. 2, the puncture device 1 includes a metallic cylindrical needle tube 3 having a longitudinal axis and a tube 5 accommodated within the needle tube 3 and composed of a cylindrical optically-transparent material. Furthermore, as shown in FIGS. 2 and 3, the puncture device 1 includes a flexible sheath 13 accommodating the needle tube 3 so as to be movable along the longitudinal axis, a substantially-cylindrical attachment adapter 15 attachable to the channel in the ultrasonic endoscope 100, an operating body 17 supported so as to be movable along the longitudinal axis relative to the attachment adapter 15, and a needle slider 19 supported so as to be movable along the longitudinal axis relative to the operating body 17.

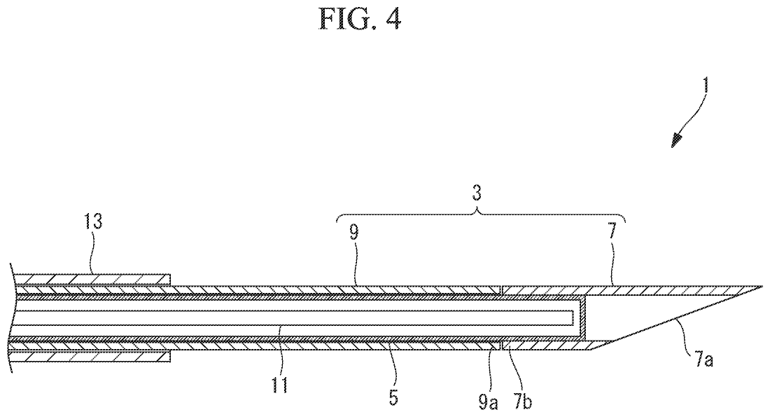

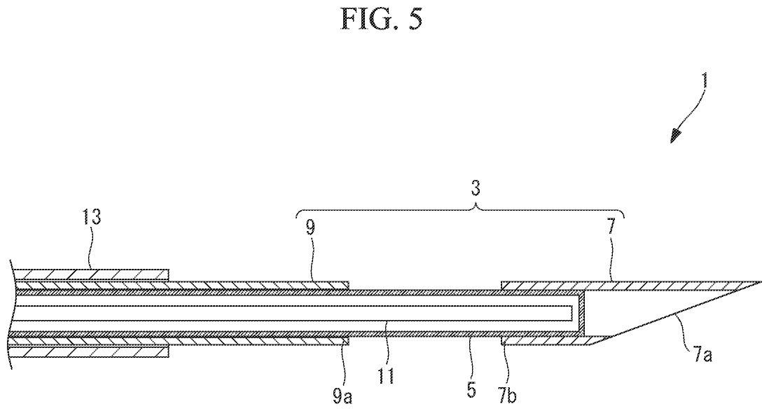

As shown in FIGS. 4 and 5, the needle tube 3 is entirely cylindrical and is constituted of a needle tip member 7 at the distal end and a needle base member 9 at the proximal end relative to the needle tip member 7.

The needle tip member 7 has a blade surface 7a obtained by diagonally cutting the tip end along a plane that intersects the longitudinal axis. The tip end of the needle tip member 7 may have an opening or may be sealed. Furthermore, in a state where a proximal end 7b of the needle tip member 7 is engaged with the distal end of the tube 5, the needle tip member 7 is fixed to the distal end of the tube 5.

The needle base member 9 is disposed at a position where it covers the proximal end of the tube 5 relative to the needle tip member 7, and is provided so as to be movable in the longitudinal direction of the tube 5. Moreover, the proximal end of the needle base member 9 extends to the needle slider 19. A distal end 9a of the needle base member 9 has an inner diameter slightly larger than and an outer diameter substantially equal to those of the proximal end 7b of the needle tip member 7.

The tube 5 is capable of accommodating an optical fiber 11 along the longitudinal axis. Furthermore, the tube 5 is transparent or white and is composed of, for example, resin that scatters or transmits light ranging between 670 nm and 850 nm. The proximal end of the tube 5 extends to the needle slider 19. The distal end of the tube 5 may have an opening or may be sealed.

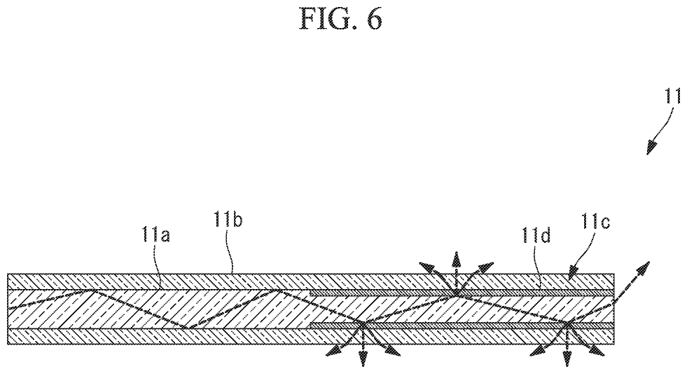

For example, as shown in FIG. 6, the optical fiber 11 includes a core layer 11a that transmits light and a cladding layer 11b that covers the outer peripheral surface of the core layer 11a. The core layer 11a and the cladding layer 11b are composed of, for example, quartz. The cladding layer 11b has an impurity added thereto for reducing the refractive index relative to that of the core layer 11a. The optical fiber 11 causes light to undergo total reflection at the boundary surface between the core layer 11a and the cladding layer 11b so as to optically guide the light. The material of the core layer 11a and the cladding layer 11b is not limited to quartz and may be, for example, a translucent resin.

The distal end of the optical fiber 11 is provided with a light radiation section (i.e., a light radiation range) 11c that radiates light from the side surface of the optical fiber 11 by causing a portion of light optically guided from the proximal end toward the distal end of the core layer 11a to be transmitted through the cladding layer 11b without being reflected at the boundary surface between the core layer 11a and the cladding layer 11b. For example, the light radiation section 11c has a predetermined length from the distal end toward the proximal end of the optical fiber 11.

The light radiation section 11c is constituted of a scattering agent (i.e., resin or glass particles) 11d applied to a part of the core layer 11a or to the boundary between the core layer 11a and the cladding layer 11b. The portion of light optically guided from the proximal end of the core layer 11a can be scattered radially outward from the optical fiber 11 by the light radiation section 11c.

As shown in FIG. 2, the proximal end of the sheath 13 extends to the operating body 17.

The needle slider 19 includes a slider body 21 having a longitudinal axis extending in the longitudinal direction of the sheath 13, and also includes a pipe sliding section (i.e., a needle-tube operating section) 29 supported so as to be movable along the longitudinal axis relative to the slider body 21.

As shown in FIGS. 2 and 3, the slider body 21 includes, in the following order from the distal end, a cylindrical tube 23 capable of accommodating the operating body 17, a rail 25 extending linearly along the longitudinal axis from the proximal end of the tube 23, and a proximal end 27 fixed to the proximal end of the rail 25.

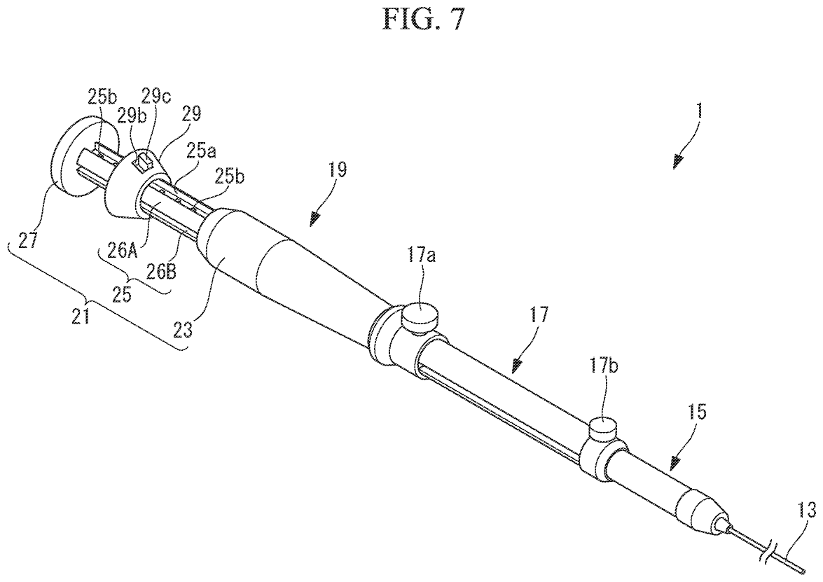

For example, as shown in FIGS. 3 and 7, the rail 25 is constituted of two rail members 26A and 26B extending along the longitudinal axis. The two rail members 26A and 26B are disposed parallel to each other with a distance therebetween in a direction orthogonal to the longitudinal axis. The outer surface of the rail member 26A is provided with a rail groove 25a extending along the longitudinal axis.

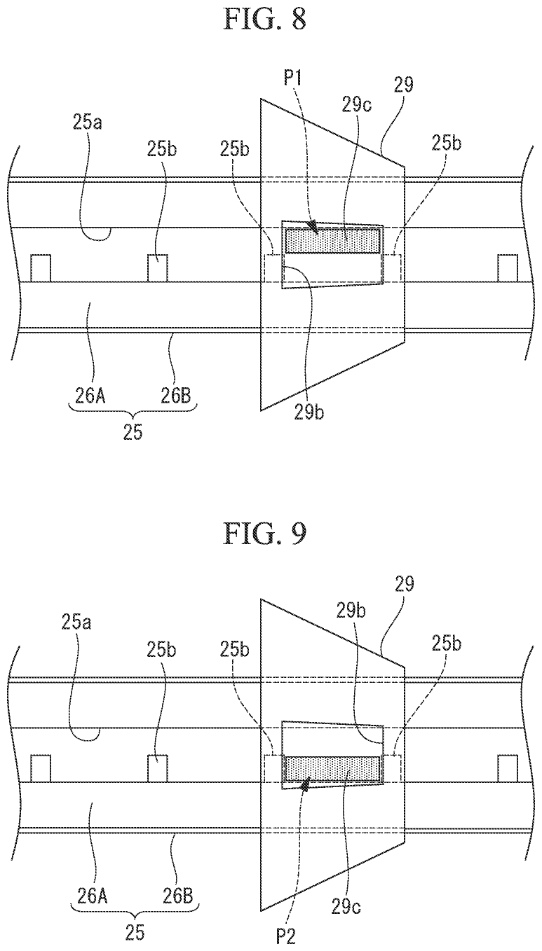

For example, as shown in FIGS. 8 and 9, the rail groove 25a is provided with a plurality of protrusions (i.e., a positioning mechanism) 25b that are disposed on one of the inner side surfaces in the widthwise direction and that are arranged with a predetermined distance therebetween in the longitudinal direction. Each protrusion 25b protrudes in the widthwise direction of the rail groove 25a and has a fixed gap relative to the opposite inner side surface in the widthwise direction.

As shown in FIG. 2, the proximal end of the tube 5 is fixed to the proximal end 27 of the slider body 21. By moving the needle slider 19 along the longitudinal axis relative to the operating body 17, the tube 5 is advanced and retracted in the longitudinal direction relative to the sheath 13 fixed to the operating body 17.

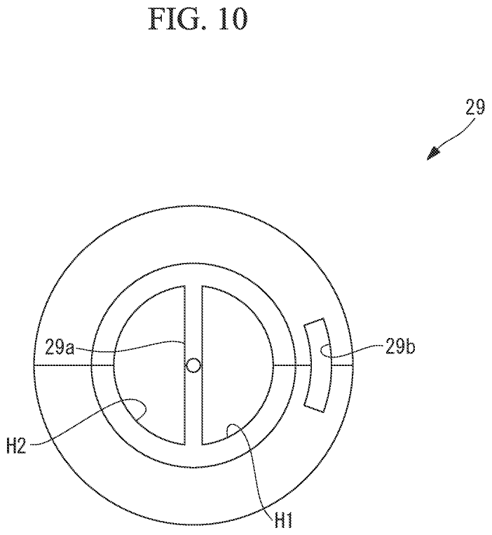

For example, as shown in FIGS. 7 and 10, the pipe sliding section 29 is tubular and has a substantially circular truncated cone shape whose diameter gradually increases from the distal end toward the proximal end in the axial direction. The pipe sliding section 29 is provided with a wall-like partition 29a that partitions the internal space into two spaces along the central axis of the pipe sliding section 29. The two spaces will be referred to as internal spaces H1 and H2 hereinafter.

In the pipe sliding section 29, the rail member 26A of the rail 25 extends through the internal space H1, and the rail member 26B of the rail 25 extends through the internal space H2. The partition 29a is disposed in a gap between the rail member 26A and the rail member 26B. Accordingly, the pipe sliding section 29 is provided so as to be movable in the longitudinal direction of the rail 25 while being positioned in the radial direction.

In the pipe sliding section 29, the proximal end of the needle base member 9 is fixed on the central axis of the partition 29a. When the pipe sliding section 29 is moved along the rail 25, the needle base member 9 of the needle tube 3 is advanced and retracted in the longitudinal direction relative to the tube 5 fixed to the proximal end 27 of the slider body 21.

When the needle base member 9 of the needle tube 3 is advanced in the longitudinal direction relative to the tube 5, for example, as shown in FIG. 4, the distal end 9a of the needle base member 9 abuts on the proximal end 7b of the needle tip member 7, whereby the needle tip member 7 becomes supported by the needle base member 9. Accordingly, the pushability required for puncturing, that is, the rigidity of the needle tube 3 for transmitting a force from the proximal end to the distal end of the needle tube 3, can be ensured.

On the other hand, when the needle base member 9 of the needle tube 3 is retracted in the longitudinal direction relative to the tube 5, for example, as shown in FIG. 5, the distal end 9a of the needle base member 9 is positioned away from the proximal end 7b of the needle tip member 7 in the longitudinal direction, so that the tube 5 composed of an optically transparent material and covered with the needle base member 9 becomes partially exposed. Accordingly, light emitted from the light radiation section 11c of the optical fiber 11 and transmitted through an exposed region of the tube 5 can be radiated from around the needle tube 3.

Furthermore, as shown in FIGS. 3 and 7, the pipe sliding section 29 has a lock button (i.e., a positioning mechanism) 29c fitted within a window frame 29b provided in the side surface of the pipe sliding section 29. The lock button 29c is disposed in the rail groove 25a of the rail member 26A and is movable in the widthwise direction of the rail groove 25a.

When the lock button 29c is moved to an unlocking position P1 shown in FIG. 8, that is, a position opposite the protrusions 25b in the widthwise direction of the rail groove 25a, the lock button 29c can move along the longitudinal axis within the rail groove 25a without being locked between the protrusions 25b. Accordingly, the pipe sliding section 29 is allowed to move in the longitudinal direction of the rail 25.

On the other hand, when the lock button 29c is moved to a locking position P2 shown in FIG. 9, that is, a position toward the protrusions 25b in the widthwise direction of the rail groove 25a, the lock button 29c becomes locked between the protrusions 25b. Accordingly, the pipe sliding section 29 is restricted from moving in the longitudinal direction of the rail 25.

For example, the protrusions 25b are disposed at a position where the distal end 9a of the needle base member 9 is positioned in abutment with the proximal end 7b of the needle tip member 7 and a position where the distal end 9a of the needle base member 9 is positioned away from the proximal end 7b of the needle tip member 7 in the longitudinal direction by a predetermined distance.

The proximal end of the sheath 13 is fixed to the operating body 17. By moving the operating body 17 along the longitudinal axis relative to the attachment adapter 15, the sheath 13 fixed to the operating body 17 is advanced and retracted relative to the attachment adapter 15 along the longitudinal axis together with the tube 5 fixed to the needle slider 19 and the needle tube 3 fixed to the pipe sliding section 29.

Furthermore, when the needle slider 19 is moved along the longitudinal axis relative to the operating body 17, the tube 5 fixed to the proximal end 27 of the needle slider 19 is advanced and retracted relative to the sheath 13 fixed to the operating body 17 along the longitudinal axis together with the needle tube 3 fixed to the pipe sliding section 29.

Furthermore, as shown in FIGS. 2, 3, and 7, the operating body 17 is provided with a stopper 17a that defines the advanced position of the needle slider 19 in an adjustable manner relative to the operating body 17, and a fastening screw 17b for fixing the operating body 17 at a freely-chosen position relative to the attachment adapter 15. In FIG. 2, a groove provided for preventing the stopper 17a from rotating relative to the longitudinal axis of the operating body 17 is not shown.

Next, a phototherapy method using the puncture device 1 according to this embodiment will be described below.

The following description relates to an example where the phototherapy method according to this embodiment is applied to the cancer cells Z existing in the pancreas Y, as shown in FIG. 1.

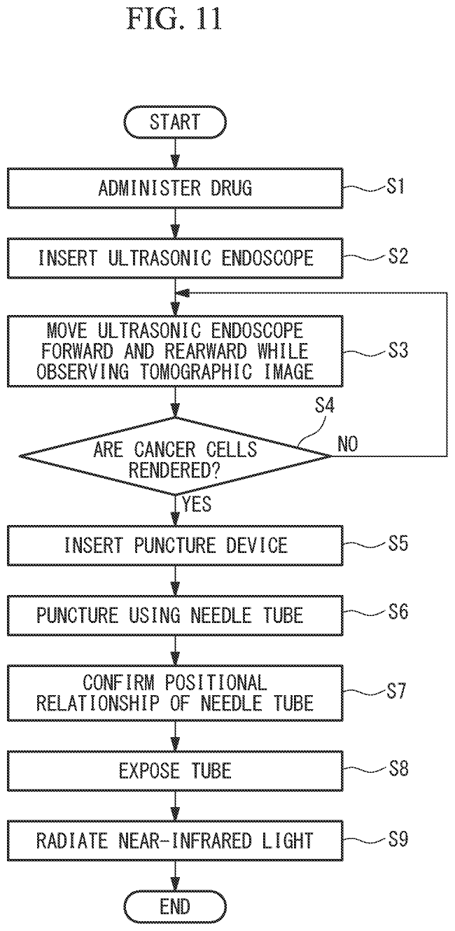

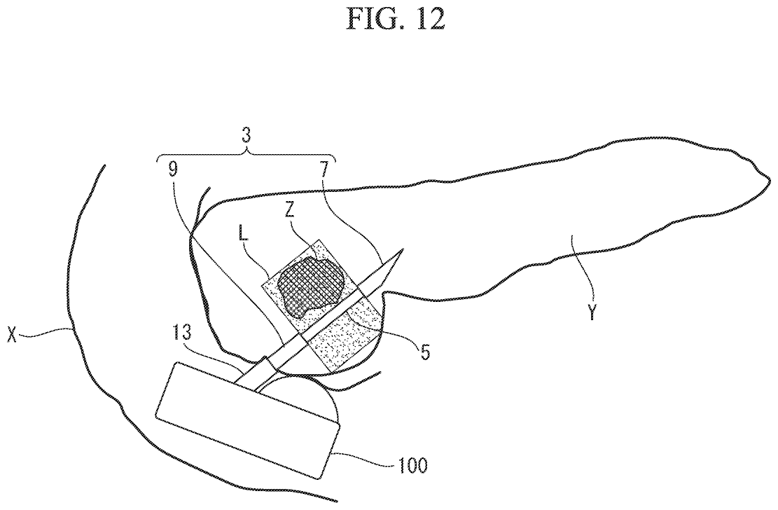

As shown in a flowchart in FIG. 11, the phototherapy method according to this embodiment involves preliminarily administering, to a patient, a drug that kills the cancer cells (i.e., an irradiation site) Z by reacting to near-infrared light L (see FIG. 12) (step S1), and inserting the ultrasonic endoscope 100 into the alimentary canal X, such as the stomach or the duodenum (step S2).

While the cancer cells Z in a tomographic image of the pancreas Y located near the alimentary canal X are being observed through the ultrasonic endoscope 100, the ultrasonic endoscope 100 is moved forward or rearward (step S3), and the ultrasonic endoscope 100 is disposed at a position where the cancer cells Z can be rendered (step S4).

Once the ultrasonic endoscope 100 is disposed at the position where the cancer cells Z can be rendered, the puncture device 1 positioned in a state where the distal end 9a of the needle base member 9 of the needle tube 3 is in abutment with the proximal end 7b of the needle tip member 7 is inserted into the body via the channel in the ultrasonic endoscope 100, as shown in FIG. 4 (step S5). At this time, the attachment adapter 15 of the puncture device 1 is attached to the channel in the ultrasonic endoscope 100.

Subsequently, as shown in FIG. 1, the needle tube 3 of the puncture device 1 is caused to protrude from the opening at the distal end of the channel in the ultrasonic endoscope 100, so that the needle tube 3 punctures the canal wall of the alimentary canal X. Then, the needle tube 3 penetrates through the canal wall of the alimentary canal X, so that the needle tube 3 punctures the pancreas Y located near the alimentary canal X (step S6). In this case, since the needle tip member 7 having the blade surface 7a of the needle tube 3 is supported by the needle base member 9, the pushability required for puncturing can be ensured.

Furthermore, since the needle tube 3 is composed of metal, the needle tube 3 can be reliably viewed within an ultrasonic image acquired by the ultrasonic endoscope 100. A surgeon views the ultrasonic image to confirm the positional relationship between the needle tube 3 and the cancer cells Z (step S7). Specifically, before the cancer cells Z are irradiated with the near-infrared light L emitted from the optical fiber 11 within the needle tube 3, the position of the needle tube 3 relative to the cancer cells Z is confirmed in the ultrasonic image.

As shown in FIG. 12, once the cancer cells Z are disposed at a position facing the radially outer side of the needle tube 3, the pipe sliding section 29 is moved toward the proximal end relative to the slider body 21, so that the needle base member 9 of the needle tube 3 is retracted relative to the tube 5. Then, the lock button 29c is used to position the distal end 9a of the needle base member 9 away from the proximal end 7b of the needle tip member 7 in the longitudinal direction by the predetermined distance. Accordingly, as shown in FIG. 5, the tube 5 covered with the needle base member 9 becomes partially exposed (step S8).

In this case, it is preferable that the needle base member 9 positioned away from the needle tip member 7 in the longitudinal direction not be retracted to a position where the needle base member 9 falls out of the pancreas Y and that the distal end 9a of the needle base member 9 be retained in an organ, that is, the pancreas Y, having the cancer cells Z while the cancer cells Z are being irradiated with the light emitted from the optical fiber 11. With the distal end 9a of the needle base member 9 being retained in the pancreas Y, the needle tip member 7 and the needle base member 9 of the needle tube 3 are fixed to the pancreas Y. Accordingly, a force that causes the tube 5 exposed from the needle tube 3 to buckle, that is, a force acting in a direction intersecting the longitudinal axis of the needle tube 3, can be prevented from being applied to the tube 5.

Subsequently, in the state where the tube 5 is partially exposed from the needle tube 3, the near-infrared light L emitted from a light source is caused to enter the optical fiber 11. The near-infrared light L entering the optical fiber 11 propagates through the core layer 11a of the optical fiber 11 to the distal end, and is output radially in all directions from the light radiation section 11c provided at the distal end.

Because the tube 5 covering the periphery of the light radiation section 11c is exposed, the near-infrared light L output from the light radiation section 11c and transmitted through the exposed region of the tube 5 is radiated onto the cancer cells Z disposed at the radially outer side of the needle tube 3 (step S9). Accordingly, the drug preliminarily administered to the patient damages the cancer cells Z by reacting to the near-infrared light L, thereby killing the cancer cells Z.

As described above, with the puncture device 1 and the phototherapy method according to this embodiment, when the needle tube 3 is to puncture the vicinity of the cancer cells Z, the needle tip member 7 of the needle tube 3 is supported by the needle base member 9, so that the pushability required for puncturing can be ensured. Moreover, when the cancer cells Z are to be irradiated with the light from the optical fiber 11, the optical fiber 11 does not protrude from the blade surface 7a at the distal end of the needle tube 3, so that the outer surface of the optical fiber 11 can be prevented from being scraped as a result of sliding against the blade surface 7a. Consequently, breakage of the optical fiber 11 caused by the blade surface 7a of the needle tube 3 can be prevented, while the pushability of the needle tube 3 can be ensured.

This embodiment can be modified as follows.

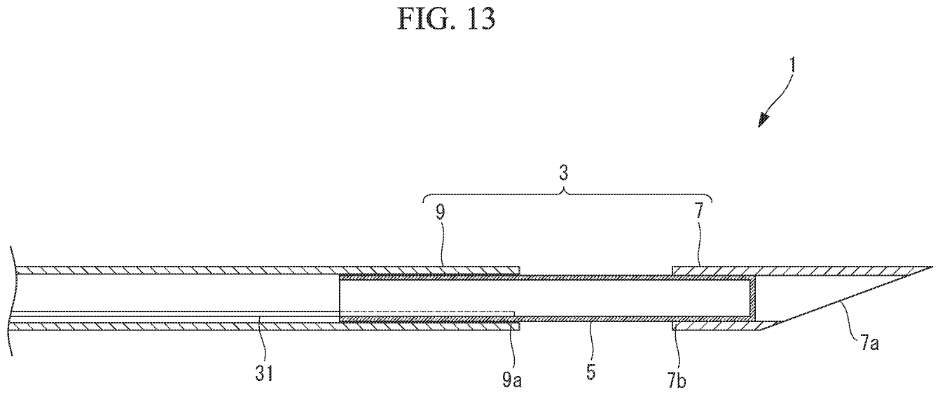

In this embodiment, the proximal end of the tube 5 is fixed to the proximal end 27 of the slider body 21. Alternatively, for example, as shown in FIG. 13, the proximal end of the tube 5 may extend to an intermediate location of the needle base member 9, and the proximal end of the tube 5 may be connected to the proximal end 27 of the slider body 21 by a metal wire (i.e., a connection member) 31.

In this case, the proximal end of the tube 5 may extend toward the proximal end relative to the position of the distal end 9a of the needle base member 9 corresponding to a case where the needle base member 9 is in a most retracted state. The metal wire 31 may extend in the longitudinal direction of the sheath 13 and may be fixed or attached to the proximal end of the tube 5 and the proximal end 27 of the slider body 21.

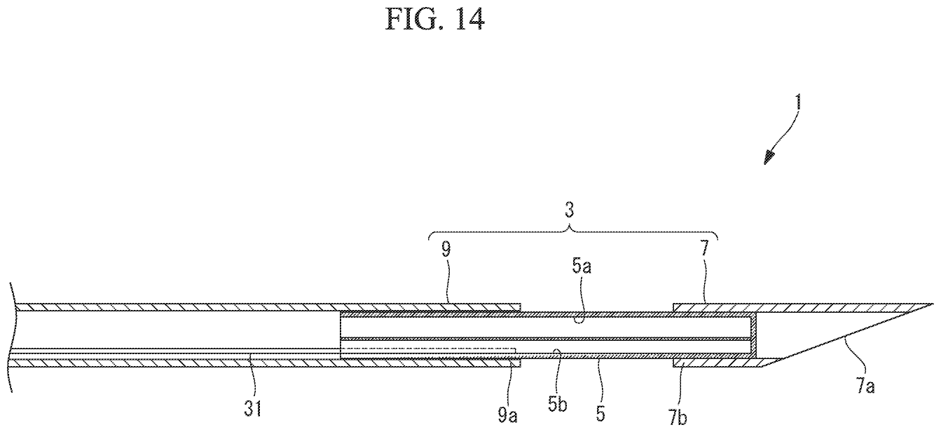

In this modification, for example, as shown in FIG. 14, the tube 5 may have two parallel lumens 5a and 5b extending in the longitudinal direction. The optical fiber 11 may be fitted in the lumen 5a so as to be extendable and retractable in the longitudinal direction, and the distal end of the metal wire 31 may be fitted in the lumen 5b. The metal wire 31 may be fixed or attached to the inner surface of the lumen 5b. With the lumen 5a that receives the optical fiber 11 and the lumen 5b that receives the metal wire 31 being separated from each other, the optical fiber 11 can be reliably prevented from breaking as a result of coming into contact with the metal wire 31.

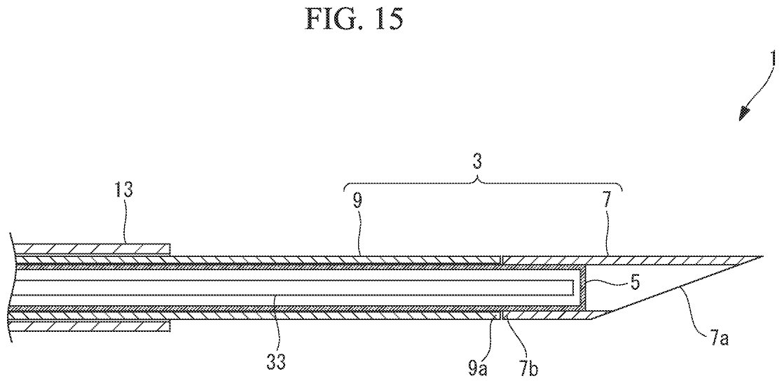

In this embodiment, the needle tube 3 punctures the vicinity of the cancer cells Z in a state where the optical fiber 11 is fitted in the tube 5. Alternatively, until the needle tube 3 punctures the cancer cells Z after the puncture device 1 is inserted into the body via the channel in the ultrasonic endoscope 100, a narrow metallic member, such as a stylet 33, may be fitted in the tube 5, as shown in FIG. 15. In this case, after the needle tube 3 punctures the vicinity of the cancer cells Z, the stylet 33 may be removed from the tube 5, and the optical fiber 11 may be fitted into the tube 5.

According to this modification, the stylet 33, which is more rigid, is fitted into the tube 5 in place of the optical fiber 11, so that the needle tube 3 can be prevented from buckling between the needle base member 9 and the needle tip member 7 when the puncture device 1 is being inserted into the channel in the ultrasonic endoscope 100 or when the needle tube 3 is puncturing the cancer cells Z.

In this embodiment, one of the outer surface of the distal end 9a of the needle base member 9 and the outer surface of the proximal end 7b of the needle tip member 7 may be provided with a tapered section or a step, and the other one of the distal end 9a of the needle base member 9 and the proximal end 7b of the needle tip member 7 may be in abutment with the tapered section or the step.

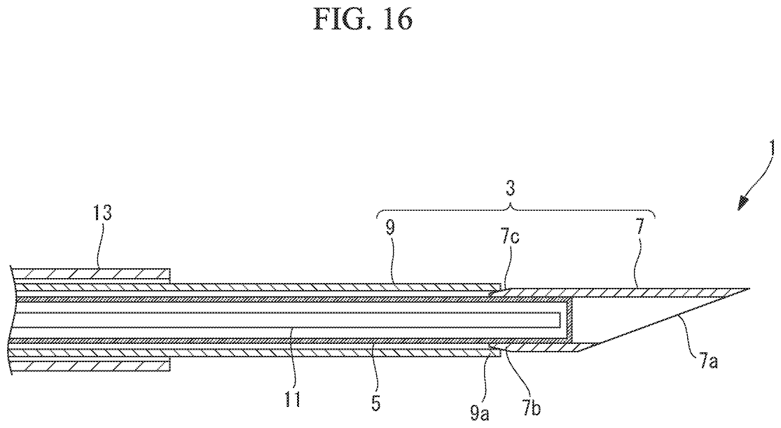

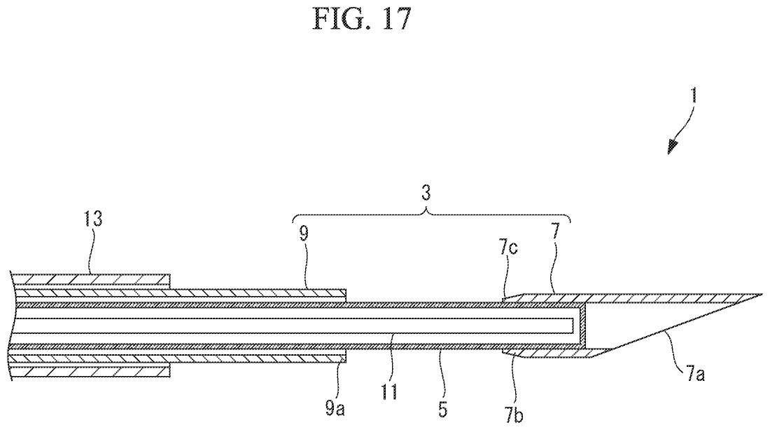

For example, in an example shown in FIGS. 16 and 17, the outer surface of the proximal end 7b of the needle tip member 7 is provided with a tapered section 7c whose outer diameter gradually decreases toward the proximal end. In a state where the distal end 9a of the needle base member 9 is in abutment with the tapered section 7c of the proximal end 7b of the needle tip member 7, the distal end 9a of the needle base member 9 covers the proximal end 7b of the needle tip member 7.

The distal end 9a of the needle base member 9 and the proximal end 7b of the needle tip member 7 overlap in the radial direction, so that the distal end 9a of the needle base member 9 and the proximal end 7b of the needle tip member 7 can be securely coupled to each other in the longitudinal direction. Accordingly, in a state where the distal end 9a of the needle base member 9 is in abutment with the proximal end 7b of the needle tip member 7, the boundary between the needle base member 9 and the needle tip member 7 is less likely to buckle.

In this modification, a step may be provided in place of the tapered section 7c. Furthermore, the outer surface of the distal end 9a of the needle base member 9 may be provided with a tapered section or a step whose outer diameter gradually decreases toward the distal end. In a state where the tapered section or the step of the distal end 9a of the needle base member 9 is in abutment with the proximal end 7b of the needle tip member 7, the distal end 9a of the needle base member 9 may cover the proximal end 7b of the needle tip member 7.

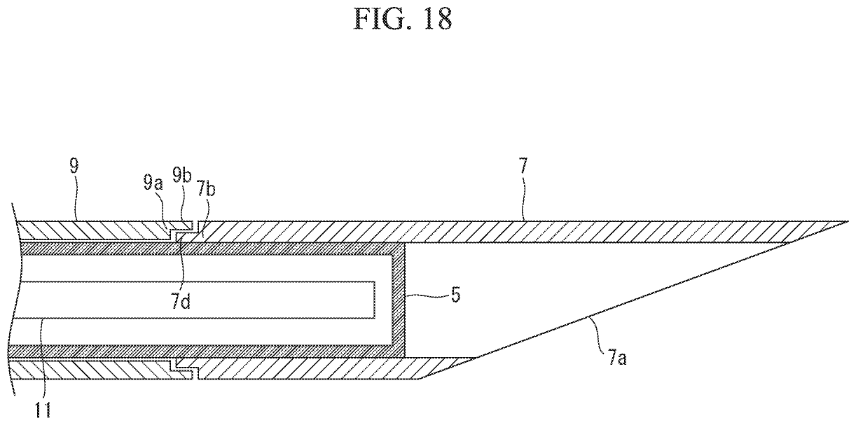

In this modification, the configuration is not limited to that described above so long as the distal end 9a and the proximal end 7b overlap in the radial direction in a state where the distal end 9a of the needle base member 9 is in abutment with the proximal end 7b of the needle tip member 7. For example, as shown in FIG. 18, the distal end 9a of the needle base member 9 may include a large diameter section 9b having a step whose inner diameter increases significantly while the outer diameter remains fixed, and the proximal end 7b of the needle tip member 7 may have a small diameter section 7d having a step whose outer diameter decreases significantly while the inner diameter remains fixed. The large diameter section 9b of the needle base member 9 may cover the small diameter section 7d of the needle tip member 7 in a state where the step on the inner surface of the large diameter section 9b of the needle base member 9 is in abutment with the step on the outer surface of the small diameter section 7d of the needle tip member 7.

With this configuration, the distal end 9a of the needle base member 9 and the proximal end 7b of the needle tip member 7 can be securely coupled to each other in the longitudinal direction while the outer surface of the distal end 9a of the needle base member 9 and the outer surface of the proximal end 7b of the needle tip member 7 are flush with each other. Alternatively, the shape of the distal end 9a of the needle base member 9 and the shape of the proximal end 7b of the needle tip member 7 may be inverted from the above configuration. In other words, the distal end 9a of the needle base member 9 may include a small diameter section, and the proximal end 7b of the needle tip member 7 may include a large diameter section.

In this embodiment, the protrusions 25b on the rail member 26A and the lock button 29c on the pipe sliding section 29 are used for positioning the distal end 9a of the needle base member 9 at two positions, namely, a position where the distal end 9a of the needle base member 9 is in abutment with the proximal end 7b of the needle tip member 7 and a position where the distal end 9a of the needle base member 9 is positioned away from the proximal end 7b of the needle tip member 7 in the longitudinal direction. The number of positions may be one or may be two or more, so long as the distal end 9a of the needle base member 9 can at least be positioned in abutment with the proximal end 7b of the needle tip member 7.

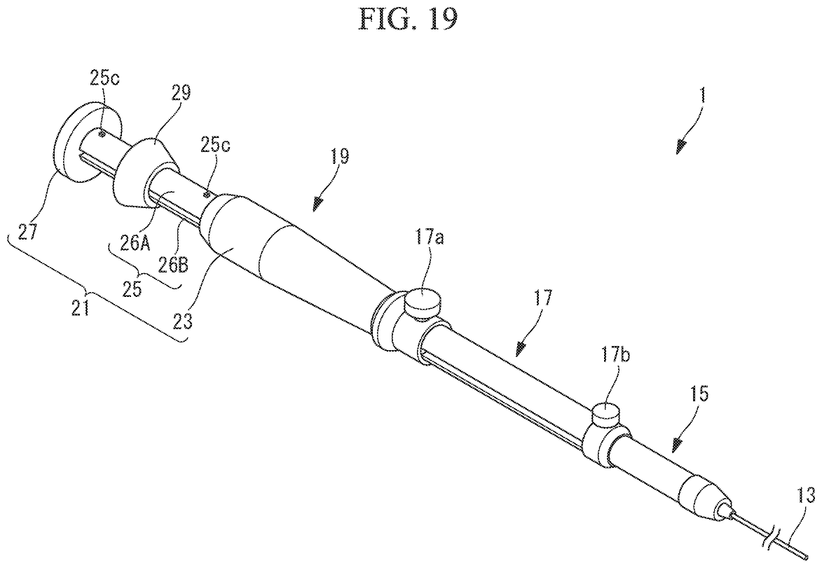

In this embodiment, the rail member 26A is provided with the rail groove 25a having the protrusions 25b, and the pipe sliding section 29 is provided with the lock button 29c. Alternatively, for example, as shown in FIG. 19, the outer surface of the rail member 26A may be provided with a plurality of protrusions (i.e., a positioning mechanism) 25c arranged with a predetermined distance therebetween in the longitudinal direction, and the inner surface of the pipe sliding section 29 may be provided with a recess (not shown) engageable with one of the protrusions 25c on the rail member 26A. The needle base member 9 may be positioned relative to the tube 5 in the longitudinal direction by engaging one of the protrusions 25c on the rail member 26A with the recess in the pipe sliding section 29.

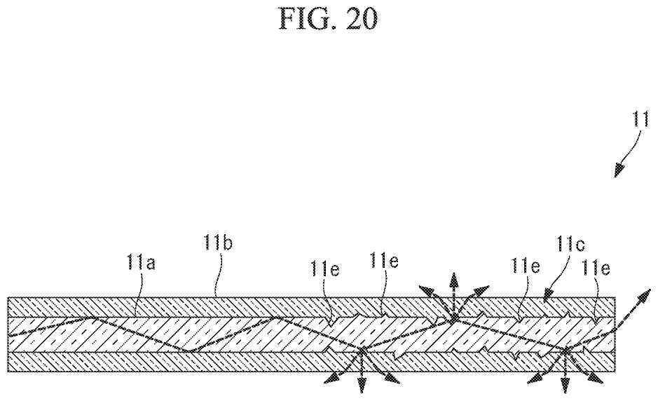

In this embodiment, the light radiation section 11c of the optical fiber 11 is constituted of the scattering agent 11d. The light radiation section 11c may be of any type so long as it can cause a portion of light optically guided from the proximal end of the core layer 11a to be scattered radially outward from the optical fiber 11. For example, as shown in FIG. 20, the light radiation section 11c may be constituted of a plurality of small protrusions and recesses 11e provided at the boundary surface between the core layer 11a and the cladding layer 11b.

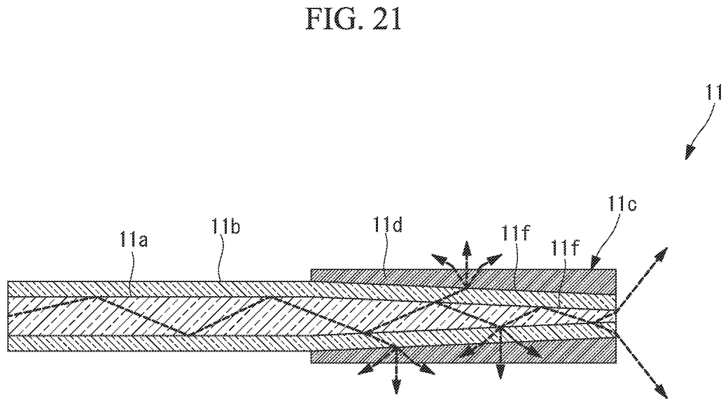

For example, as shown in FIG. 21, the light radiation section 11c may be constituted of a tapered section 11f provided at the distal end of both the core layer 11a and the cladding layer 11b and the scattering agent 11d applied to the distal end of the cladding layer 11b or to the surface of the distal end of the cladding layer 11b. With the core layer 11a and the cladding layer 11b being tapered gradually toward the distal end, the conditions for total reflection can be abolished, the portion of the light optically guided from the proximal end can be made to leak toward the side surface, and the light can be effectively scattered radially outward from the optical fiber 11 by the scattering agent 11d.

Moreover, the light radiation section 11c may be constituted by partially removing the cladding layer 11b from the distal end 9a to expose the core layer 11a.

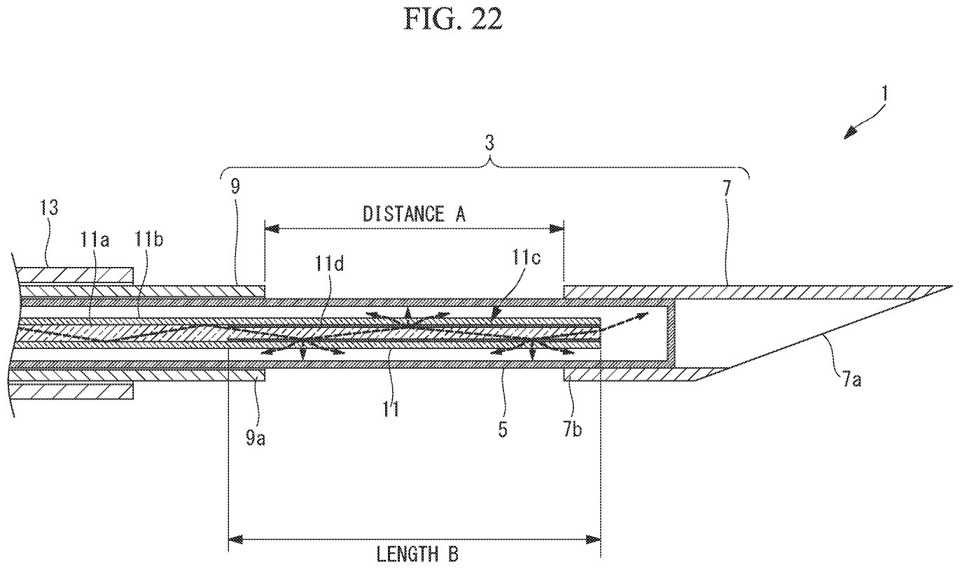

In this embodiment and the modifications, it is preferable that the relationship between a distance A of a range in which the tube 5 is partially exposed from the needle tube 3 and a length B of the light radiation section 11c of the optical fiber 11 be set as follows. For example, the pipe sliding section 29 is moved maximally toward the proximal end relative to the slider body 21. In other words, the pipe sliding section 29 is moved until the proximal end surface thereof abuts on the proximal end 27 of the slider body 21. In this state, for example, as shown in FIG. 22, the length B from the distal end to the proximal end of the light radiation section 11c may be greater than the distance A between the distal end of the needle base member 9 and the proximal end of the needle tip member 7.

In this case, the distal end of the light radiation section 11c may be positioned toward the distal end of the needle tube 3 relative to the proximal end of the needle tip member 7. Moreover, the proximal end of the light radiation section 11c may be positioned toward the proximal end of the needle tube 3 relative to the position of the distal end of the needle base member 9 when the proximal end surface of the pipe sliding section 29 is in abutment with the proximal end 27 of the slider body 21. With this configuration, the portion of light optically guided from the proximal end of the core layer 11a of the optical fiber 11 can be reliably scattered radially outward from the needle tube 3.

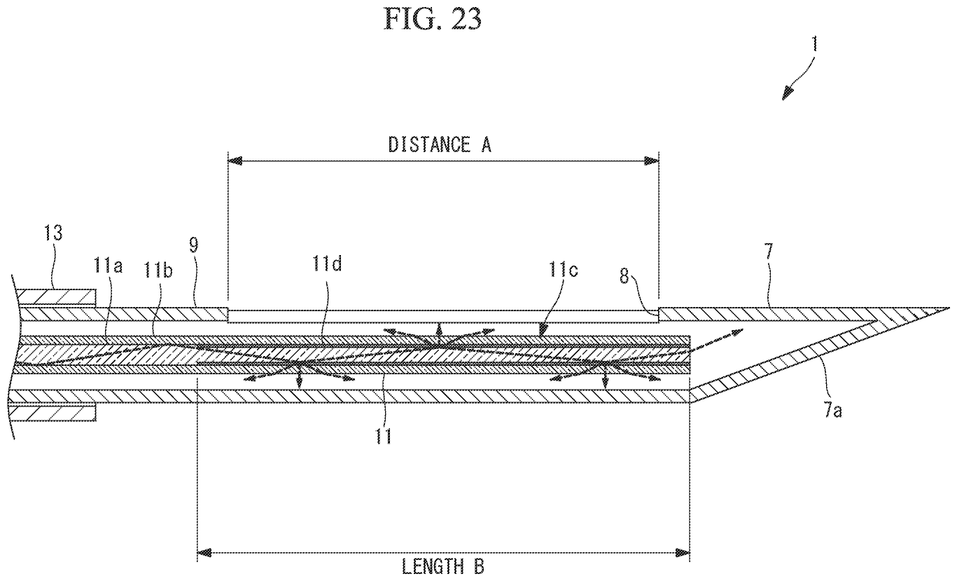

The relationship between the distance A of the range in which the tube 5 is partially exposed and the length B of the light radiation section 11c does not necessarily have to be applied to the case where the needle base member 9 slides relative to the tube 5. For example, as shown in FIG. 23, the relationship may be applied to a type having a side hole 3a extending through the distal end of the needle tube 3 in the direction intersecting the longitudinal axis, that is, a type in which the needle base member 9 does not slide relative to the tube 5.

In this case, the distance A between the distal end and the proximal end of the side hole 3a in the longitudinal direction of the needle tube 3 may be greater than the length B from the distal end to the proximal end of the light radiation section 11c of the optical fiber 11. Furthermore, the distal end of the light radiation section 11c may be positioned toward the distal end of the needle tube 3 relative to the distal end of the side hole 3a. Moreover, the proximal end of the light radiation section 11c may be positioned toward the proximal end of the needle tube 3 relative to the proximal end of the side hole 3a.

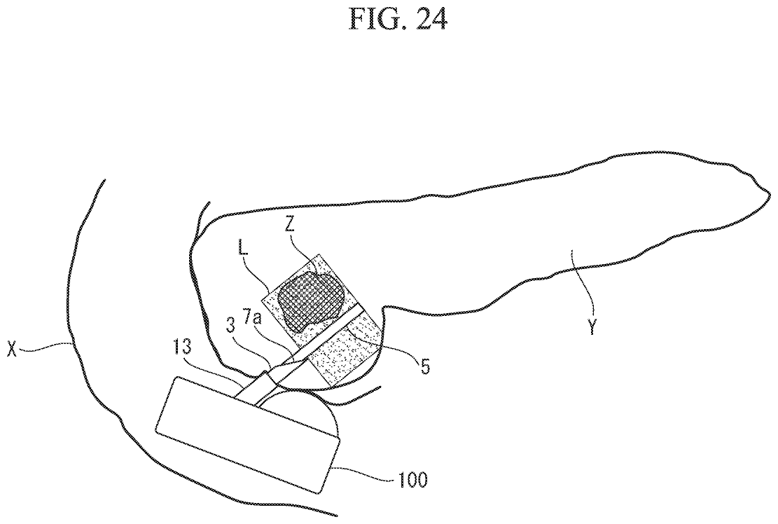

In this embodiment, the needle tube 3 is separated into the needle tip member 7 and the needle base member 9. Alternatively, for example, as shown in FIG. 24, the tube 5 accommodating the optical fiber 11 may be exposed from the distal end of the needle tube 3. In this case, the blade surface 7a at the distal end of the needle tube 3 may have an opening. Furthermore, the tube 5 may be exposed along the longitudinal axis from the blade surface 7a of the needle tube 3 by pulling the needle tube 3 toward oneself relative to the tube 5.

In this modification, it is similarly preferable that the needle tip not be retracted to a position where the needle tip falls out of the pancreas Y and that the needle tip be retained in an organ, that is, the pancreas Y, having the cancer cells Z while the cancer cells Z in the pancreas Y are being irradiated with the light emitted from the optical fiber 11. Accordingly, a force that causes the tube 5 exposed from the needle tube 3 to buckle or a force that causes the optical fiber 11 to bend, that is, a force acting in the direction intersecting the longitudinal axis of the needle tube 3, can be prevented from being applied to the optical fiber 11.

As a result, the above-described embodiment leads to the following aspects.

A first aspect of the present invention is directed to a puncture device comprising: a metallic needle tube having a longitudinal axis; and a tube that is accommodated within the needle tube, the tube being capable of accommodating an optical fiber along the longitudinal axis, the tube being composed of a cylindrical optically-transparent material. The needle tube comprises a needle tip member and a needle base member. The needle tip member is fixed to a distal end of the tube and has a blade surface at a tip of the needle tip member. The needle base member is disposed at a position where the needle base member covers a proximal end of the tube relative to the needle tip member and is movable along the longitudinal axis. The needle base member is advanced to cause a distal end of the needle base member to abut on a proximal end of the needle tip member, and the needle base member is retracted to cause the distal end of the needle base member to move away from the proximal end of the needle tip member in a direction of the longitudinal axis.

According to this aspect, the tube accommodating the optical fiber along the longitudinal axis is accommodated within the metallic needle tube. In a state where the needle base member of the needle tube is advanced to bring the distal end of the needle base member into abutment with the proximal end of the needle tip member, the needle tip member is supported by the needle base member. Accordingly, the pushability required for puncturing, that is, the rigidity of the needle tube for transmitting a force from the proximal end to the distal end of the needle tube, can be ensured. On the other hand, when the needle base member of the needle tube is retracted to move the distal end of the needle base member away from the proximal end of the needle tip member in the direction of the longitudinal axis, the tube composed of the optically transparent material and covered with the needle base member becomes partially exposed. Accordingly, light emitted from the optical fiber and transmitted through the exposed region of the tube can be radiated from around the needle tube.

Consequently, when the needle tube is to puncture the vicinity of the irradiation site, the needle tip member of the needle tube is supported by the needle base member, so that the pushability required for puncturing can be ensured. Moreover, when the irradiation site is to be irradiated with the light from the optical fiber, the optical fiber does not protrude from the blade surface at the distal end of the needle tube, so that the outer surface of the optical fiber can be prevented from being scraped as a result of sliding against the blade surface. Consequently, breakage of the optical fiber caused by the blade surface of the needle tube can be prevented, while the pushability of the needle tube can be ensured.

In the above aspect, the puncture device may further comprise: a sheath that accommodates the needle tube so as to be movable along the longitudinal axis; an operating body fixed to a proximal end of the sheath; and a needle slider that is supported so as to be movable along the longitudinal axis relative to the operating body, the needle slider being connected to the tube.

According to this configuration, when the needle slider is moved along the longitudinal axis relative to the operating body, the needle tip member of the needle tube fixed to the distal end of the tube can be advanced and retracted in the direction of the longitudinal axis relative to the sheath.

In the above aspect, the puncture device may further include: a sheath that accommodates the needle tube so as to be movable along the longitudinal axis; an operating body fixed to a proximal end of the sheath; a needle slider supported so as to be movable along the longitudinal axis relative to the operating body; and a connection member that connects the needle slider and the tube.

According to this configuration, when the needle slider is moved along the longitudinal axis relative to the operating body, the connection member is pushed and pulled in the direction of the longitudinal axis, so that a pushing force and a pulling force are transmitted to the tube. Consequently, the needle tip member of the needle tube fixed to the distal end of the tube can be advanced and retracted in the direction of the longitudinal axis relative to the sheath.

In the above aspect, the needle slider may comprise a slider body to which the proximal end of the tube is fixed and a needle-tube operating section that is supported so as to be movable along the longitudinal axis relative to the slider body and to which a proximal end of the needle base member is fixed.

According to this configuration, when the needle-tube operating section is moved along the longitudinal axis relative to the slider body, the needle base member of the needle tube can be advanced and retracted in the direction of the longitudinal axis relative to the tube.

In the above aspect, the puncture device may further include a positioning mechanism in which the slider body and the needle-tube operating section position the distal end of the needle base member in abutment with the proximal end of the needle tip member.

According to this configuration, the positioning mechanism can maintain the distal end of the needle base member in abutment with the proximal end of the needle tip member.

In the above aspect, the positioning mechanism may perform the positioning in a switchable manner between a state where the distal end of the needle base member is in abutment with the proximal end of the needle tip member and a state where the distal end of the needle base member is positioned away from the proximal end of the needle tip member in the direction of the longitudinal axis.

According to this configuration, the positioning mechanism performs the positioning in a switchable manner so as to be capable of not only maintaining the distal end of the needle base member in abutment with the proximal end of the needle tip member but also maintaining the distal end of the needle base member away from the proximal end of the needle tip member in the direction of the longitudinal axis.

In the above aspect, the distal end of the needle base member and the proximal end of the needle tip member may overlap in a radial direction in a state where the distal end of the needle base member is in abutment with the proximal end of the needle tip member.

According to this configuration, the distal end of the needle base member and the proximal end of the needle tip member can be securely coupled to each other in the direction of the longitudinal axis. Consequently, in the state where the distal end of the needle base member is in abutment with the proximal end of the needle tip member, the boundary between the needle base member and the needle tip member is less likely to buckle.

In the above aspect, a light radiation range in which light is emitted from the optical fiber may be wider in the direction of the longitudinal axis than a range in which the tube is exposed in a state where the distal end of the needle base member is positioned maximally away from the proximal end of the needle tip member in the direction of the longitudinal axis.

According to this configuration, the portion of light optically guided by the optical fiber can be reliably scattered radially outward from the needle tube.

In the above aspect, in the state where the distal end of the needle base member is positioned maximally away from the proximal end of the needle tip member in the direction of the longitudinal axis, a distal end of the light radiation range in the direction of the longitudinal axis may be positioned toward the distal end of the needle tube relative to the proximal end of the needle tip member and a proximal end of the light radiation range in the direction of the longitudinal axis may be positioned toward the proximal end of the needle tube relative to the distal end of the needle base member.

A second aspect of the present invention is directed to a puncture device comprising: a metallic needle tube having a blade surface at a distal end of the needle tube; and an optical fiber accommodated within the needle tube, the optical fiber extending along a longitudinal axis of the needle tube. The distal end of the needle tube is provided with a side hole with an opening oriented in a direction intersecting the longitudinal axis and that exposes a light radiation range in which light is emitted from the optical fiber. A distal end of the light radiation range in a direction of the longitudinal axis is positioned toward the distal end of the needle tube relative to a distal end of the side hole in the direction of the longitudinal axis, and a proximal end of the light radiation range in the direction of the longitudinal axis is positioned toward a proximal end of the needle tube relative to a proximal end of the side hole in the direction of the longitudinal axis.

According to this aspect, light emitted from the light radiation range of the optical fiber can be radiated radially outward from the needle tube via the side hole in the needle tube in a state where the optical fiber is accommodated within the needle tube without protruding from the blade surface of the needle tube. Furthermore, since the distal end of the needle tube is simply provided with the side hole, the rigidity of the needle tube can be ensured. Consequently, breakage of the optical fiber caused by the blade surface of the needle tube can be prevented, while the pushability of the needle tube required for puncturing can be ensured.

A third aspect of the present invention is directed to a phototherapy method comprising: inserting an ultrasonic endoscope into an alimentary canal; rendering an irradiation site within a body by using the ultrasonic endoscope inserted in the alimentary canal; causing a needle tube accommodating a tube composed of an optically transparent material to protrude from a distal end of the ultrasonic endoscope inserted in the alimentary canal; causing the protruding needle tube to puncture a vicinity of the irradiation site; exposing a portion of the tube from the needle tube in a state where the needle tube has punctured the vicinity of the irradiation site; and transmitting light emitted from an optical fiber through the tube exposed from the needle tube to irradiate the irradiation site with the light.

In the above aspect, the needle tube may be partially retained in an organ having the irradiation site when the portion of the tube is to be exposed.

According to this configuration, the needle tip member and the needle base member of the needle tube are fixed to the organ having the irradiation site. Consequently, a force that causes the tube exposed from the needle tube, that is, a force acting in a direction intersecting the longitudinal axis of the needle tube, can be prevented from being applied to the tube.

In the above aspect, the needle tube may be separable into a needle tip member fixed to a distal end of the tube and a needle base member disposed at a position where the needle base member covers a proximal end of the tube relative to the needle tip member. The needle tube protruding from the distal end of the ultrasonic endoscope may puncture the vicinity of the irradiation site in a state where the needle tip member and the needle base member are coupled to each other in a direction of a longitudinal axis of the needle tube. The portion of the tube may be exposed from the needle tube by moving the needle base member away from the needle tip member in the direction of the longitudinal axis in a state where the needle tube has punctured the vicinity of the irradiation site.

In the above aspect, the needle base member may be retained in an organ having the irradiation site while the irradiation site is being irradiated with the light emitted from the optical fiber.

In the above aspect, a needle tip of the needle tube may be retained in the organ having the irradiation site while the irradiation site is being irradiated with the light emitted from the optical fiber.

The present invention is advantageous in that it can prevent breakage of an optical fiber caused by a blade surface of a needle tube while maintaining an ability to puncture a target organ.

### REFERENCE SIGNS LIST

1 puncture device
3 needle tube
5 tube
7 needle tip member
7a blade surface
7b proximal end
9 needle base member
9a distal end
13 sheath
17 operating body
19 needle slider
21 slider body
25b protrusion (positioning mechanism)
25c protrusion (positioning mechanism)
29 pipe sliding section (needle-tube operating section)
29c lock button (positioning mechanism)
31 metal wire (connection member)
Z cancer cells (irradiation site)

1. A puncture device comprising:
a metallic needle tube having a longitudinal axis; and
a tube accommodated within the needle tube, the tube being capable of accommodating an optical fiber along the longitudinal axis, the tube being composed of a cylindrical optically-transparent material,
wherein the needle tube comprises a needle tip member and a needle base member, the needle tip member being fixed to a distal end of the tube and having a blade surface at a tip of the needle tip member, the needle base member being disposed at a position where the needle base member covers a proximal end of the tube relative to the needle tip member and being movable along the longitudinal axis, and
wherein the needle base member advances to cause a distal end of the needle base member to abut on a proximal end of the needle tip member, and the needle base member retracts to cause the distal end of the needle base member to move away from the proximal end of the needle tip member in a direction of the longitudinal axis.

2. The puncture device according to claim 1, further comprising:
a sheath that accommodates the needle tube so as to be movable along the longitudinal axis;
an operating body fixed to a proximal end of the sheath; and
a needle slider supported so as to be movable along the longitudinal axis relative to the operating body, the needle slider being connected to the tube.

3. The puncture device according to claim 1, further comprising:
a sheath that accommodates the needle tube so as to be movable along the longitudinal axis;
an operating body fixed to a proximal end of the sheath;
a needle slider supported so as to be movable along the longitudinal axis relative to the operating body; and
a connection member that connects the needle slider and the tube.

4. The puncture device according to claim 2,
wherein the needle slider comprises a slider body to which the proximal end of the tube is fixed and a needle-tube operating section that is supported so as to be movable along the longitudinal axis relative to the slider body and to which a proximal end of the needle base member is fixed.

5. The puncture device according to claim 4, further comprising:
a positioning mechanism in which the slider body and the needle-tube operating section position the distal end of the needle base member in abutment with the proximal end of the needle tip member.

6. The puncture device according to claim 5,
wherein the positioning mechanism performs the positioning in a switchable manner between a state where the distal end of the needle base member is in abutment with the proximal end of the needle tip member and a state where the distal end of the needle base member is positioned away from the proximal end of the needle tip member in the direction of the longitudinal axis.

7. The puncture device according to claim 1,
wherein the distal end of the needle base member and the proximal end of the needle tip member overlap in a radial direction in a state where the distal end of the needle base member is in abutment with the proximal end of the needle tip member.

8. The puncture device according to claim 1,
wherein a light radiation range in which light is emitted from the optical fiber is wider in the direction of the longitudinal axis than a range in which the tube is exposed in a state where the distal end of the needle base member is positioned maximally away from the proximal end of the needle tip member in the direction of the longitudinal axis.

9. The puncture device according to claim 8,
wherein, in the state where the distal end of the needle base member is positioned maximally away from the proximal end of the needle tip member in the direction of the longitudinal axis, a distal end of the light radiation range in the direction of the longitudinal axis is positioned toward the distal end of the needle tube relative to the proximal end of the needle tip member and a proximal end of the light radiation range in the direction of the longitudinal axis is positioned toward the proximal end of the needle tube relative to the distal end of the needle base member.

10. A puncture device comprising:
a metallic needle tube having a blade surface at a distal end of the needle tube; and
an optical fiber accommodated within the needle tube, the optical fiber extending along a longitudinal axis of the needle tube,
wherein the distal end of the needle tube is provided with a side hole with an opening oriented in a direction intersecting the longitudinal axis and that exposes a light radiation range in which light is emitted from the optical fiber, and
wherein a distal end of the light radiation range in a direction of the longitudinal axis is positioned toward the distal end of the needle tube relative to a distal end of the side hole in the direction of the longitudinal axis, and a proximal end of the light radiation range in the direction of the longitudinal axis is positioned toward a proximal end of the needle tube relative to a proximal end of the side hole in the direction of the longitudinal axis.

11. A phototherapy method comprising:
inserting an ultrasonic endoscope into an alimentary canal;
rendering an irradiation site within a body by using the ultrasonic endoscope inserted in the alimentary canal;
causing a needle tube accommodating a tube composed of an optically transparent material to protrude from a distal end of the ultrasonic endoscope inserted in the alimentary canal;
causing the protruding needle tube to puncture a vicinity of the irradiation site;
exposing a portion of the tube from the needle tube in a state where the needle tube has punctured the vicinity of the irradiation site; and
transmitting light emitted from an optical fiber through the tube exposed from the needle tube to irradiate the irradiation site with the light.

12. The phototherapy method according to claim 11,
wherein the needle tube is partially retained in an organ having the irradiation site when the portion of the tube is to be exposed.

13. The phototherapy method according to claim 11,
wherein the needle tube is separable into a needle tip member fixed to a distal end of the tube and a needle base member disposed at a position where the needle base member covers a proximal end of the tube relative to the needle tip member,
wherein the needle tube protruding from the distal end of the ultrasonic endoscope punctures the vicinity of the irradiation site in a state where the needle tip member and the needle base member are coupled to each other in a direction of a longitudinal axis of the needle tube, and
wherein the portion of the tube is exposed from the needle tube by moving the needle base member away from the needle tip member in the direction of the longitudinal axis in a state where the needle tube has punctured the vicinity of the irradiation site.

14. The phototherapy method according to claim 13,
wherein the needle base member is retained in an organ having the irradiation site while the irradiation site is being irradiated with the light emitted from the optical fiber.

15. The phototherapy method according to claim 12,
wherein a needle tip of the needle tube is retained in the organ having the irradiation site while the irradiation site is being irradiated with the light emitted from the optical fiber.

16. The puncture device according to claim 3,
wherein the needle slider comprises a slider body to which the proximal end of the tube is fixed and a needle-tube operating section that is supported so as to be movable along the longitudinal axis relative to the slider body and to which a proximal end of the needle base member is fixed.

17. The puncture device according to claim 16, further comprising:
a positioning mechanism in which the slider body and the needle-tube operating section position the distal end of the needle base member in abutment with the proximal end of the needle tip member.

18. The puncture device according to claim 17,
wherein the positioning mechanism performs the positioning in a switchable manner between a state where the distal end of the needle base member is in abutment with the proximal end of the needle tip member and a state where the distal end of the needle base member is positioned away from the proximal end of the needle tip member in the direction of the longitudinal axis.
